(19) United States
(12) Patent Application Publication
(10) Pub. No.: US 2018/0344428 A1
(43) Pub. Date: Dec. 6, 2018

(54) SCRUB SINK AND RELATED EQUIPMENT AND METHODS

(71) Applicant: John G. Brandies, JR., Winter Park, FL (US)

(72) Inventor: John G. Brandies, JR., Winter Park, FL (US)

(21) Appl. No.: 15/829,890

(22) Filed: Dec. 2, 2017

Related U.S. Application Data
(60) Provisional application No. 62/429,172, filed on Dec. 2, 2016.

(51) Int. Cl. A61B 90/80; E03C 1/126; E03C 1/181; E03C 1/182; E03C 1/22

(52) U.S. Cl. CPC A61B 90/80; E03C 1/126; E03C 1/22; E03C 1/182; E03C 1/181

(57) ABSTRACT

A sink to reduce or eliminate infection from being transmitted may include a front wall; a back wall being opposed to the front wall; and a pair of opposing side walls connecting the front wall to the back wall. The front wall, back wall and sidewalls defining a sink cavity, and the cavity includes a drain positioned closer to the back wall than positioned to the front wall.

### FIELD OF THE INVENTION

The present invention relates to scrub sinks like those used in surgical and laboratory settings, and to related equipment and methods.

### BACKGROUND OF THE INVENTION

Scrub sinks are most frequently used in surgeries and other hospital/medical settings where cleanliness of the hands and arms is critical to infection prevention for both medical professionals and patients, although these sinks can be found in other environments where a premium is placed on hygiene and cleanliness. While properly maintained scrub sinks can be an effective defense against infection, poorly designed or maintained scrub sinks can actually be a source of infection. While routine cleaning can help mitigate this risk, further improvements are possible.

### SUMMARY OF THE INVENTION

In view of the foregoing, it is an object of the present invention to provide an improved scrub sink, as well as related equipment and methods. In general, the design and configuration of the scrub sink both inherently minimize the physical opportunities for the spread of infection and facilitate proper use by the medical professional or other user. Additionally, related equipment can routinely flush and/or sanitize the scrub sink to further minimize the possibility of infection. These and other objects, aspects and advantages of the present invention will be better appreciated in view of the drawings and following detailed description of preferred embodiments.

### BRIEF DESCRIPTION OF THE DRAWINGS

FIG. 1 is a top view of a scrub sink, according to a first embodiment of the present invention;

FIG. 2 is a front view of the scrub sink of FIG. 1;

FIG. 3 is a side view of the scrub sink of FIG. 1, with hidden components in broken lines;

FIG. 4 is a front view with the basic panels removed to show the service configuration view of the scrub sink of FIG. 1, showing internal components and associated equipment;

FIG. 5 is a sectional view taken along line S-1 of FIG. 2;

FIG. 6 is a sectional view taken along line S-2 of FIG. 2;

FIG. 7 is a sectional view taken along line S-3 of FIG. 2;

FIG. 8 is an isolated view of the sink basin and surrounding surfaces of the scrub sink of FIG. 1;

FIG. 9 is a top view of the scrub sink of FIG. 1, additionally including an eye/face wash apparatus;

FIG. 10 is a front view of the scrub sink of FIG. 9;

FIG. 11 is a side view of the scrub sink of FIG. 9;

FIG. 12 is a top view of a two station scrub sink, according to a second embodiment of the present invention;

FIG. 13 is a front view of the scrub sink of FIG. 12;

FIG. 14 is a front view with the base panels removed to show the service configuration view of the scrub sink of FIG. 12, showing internal components and associated equipment;

FIG. 15 is an isolated view of the sink basins and surrounding surfaces of the scrub sink of FIG. 12; and

FIG. 16 is a top view of the scrub sink of FIG. 12, additionally including an eye/face wash apparatus.

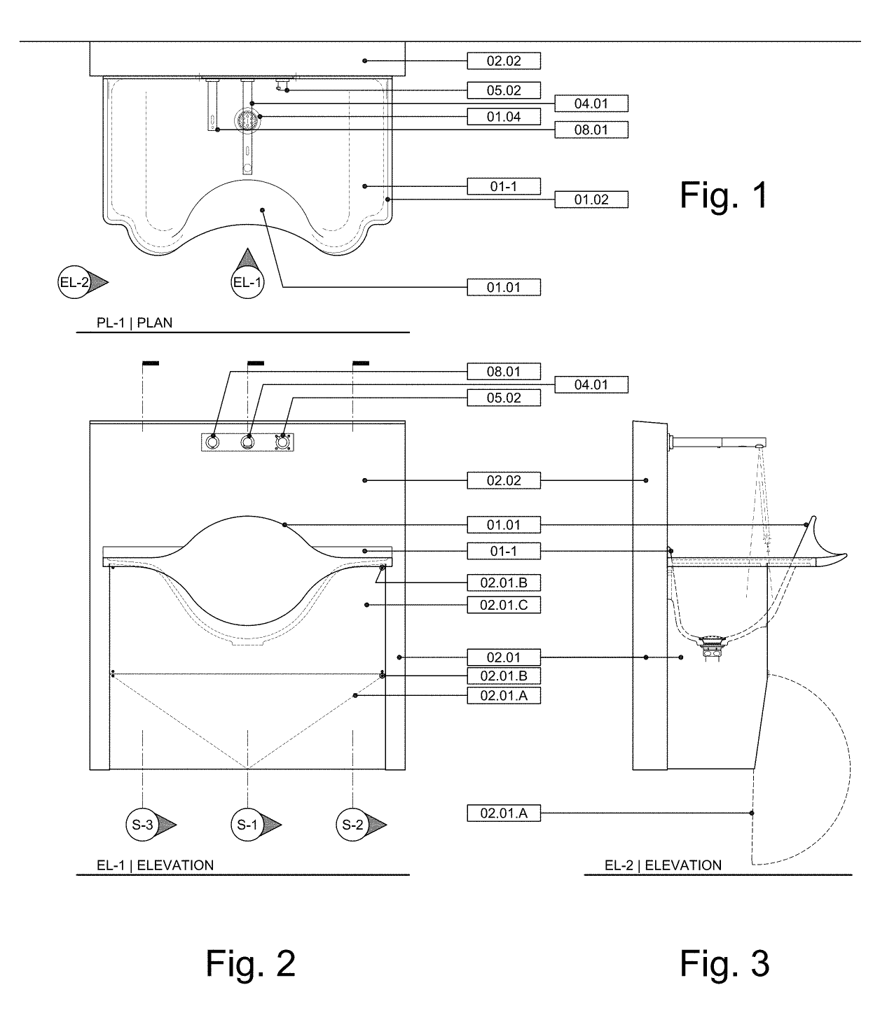

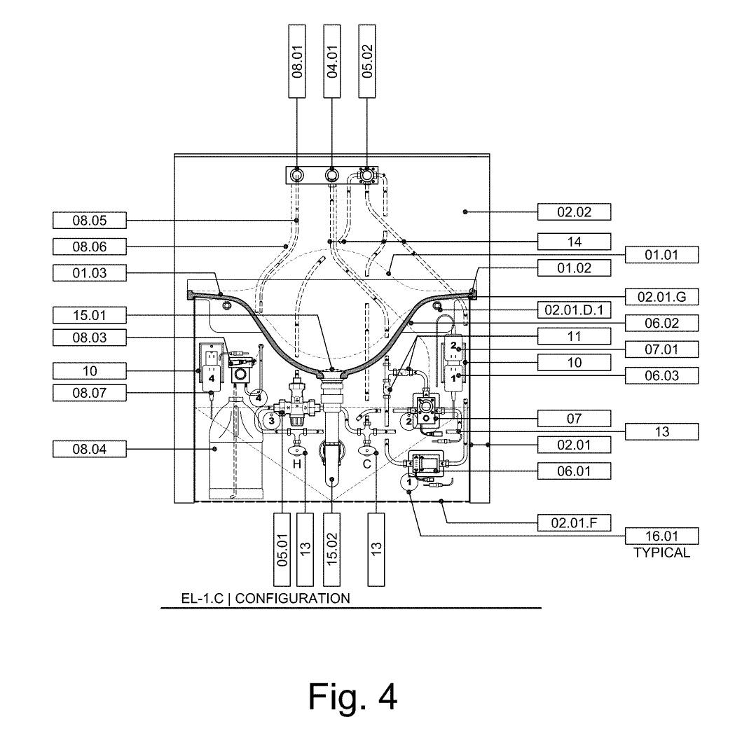

### DETAILED DESCRIPTION OF PREFERRED EMBODIMENTS

With reference to the Figures, it will be appreciated that the scrub sink is designed, as a whole, for infection control. For instance, the location of the drain is moved to the rear of the basin to allow the faucet flow to rinse and remove contamination away from the user, and to reduce back splashing that might reach the operator. All sinks have the drain located in the center of the basin, in a width direction, for equal slope to the drain from either side of the basin. The top of the basin is sloped to the drain to remove contamination. The vertical basin back wall also facilitates the direction of contaminates to the drain. The sloped basin front wall, which receives the faucet flow and hand runoff is sloped to reduce backsplashing therefrom, and also guide the water flow to the drain. The basin front rises up to form a splash guard protecting the user from the contamination in the sink basin.

Since bacteria and other infectious contaminants tend to collect in the drain, a purposeful misalignment between the drain and faucet prevents faucet water from impinging on and splashing out of the drain. In addition to preventing drain impingement/splashing, a forward faucet location assists the person washing and rinse their hands and arms. If the faucet nozzle is not adequately long, the user cannot easily rinse their forearms.

The faucet rinse is placed forward of the drain and not directly over the drain. The placement of the rinse forward to the user allows for the rinse cycle to capture containments and then move the contamination away from the user and into the drain.

The faucet spout nozzle is advantageously a non-aerating laminar flow device. Laminar flow removes turbulence from within the water column allowing the water droplets to maintain their natural molecular cohesion and therefore reduce splashing and the spread of infection, and the non-aerating portion of the nozzle removes air that would otherwise be entrained through the spout nozzle, and with removing other biological matter may be present in and/or sustained by the entrained air.

The scrub sink includes electronic controls to automatically flush the faucet with water at set time intervals to remove any water remaining with the faucet. This helps eliminate the possibility of pathogens within the faucet.

The scrub sink further includes a hygienic thermal flush system that will purge the system with scalding water for the time required to eliminate pathogens as the length of time is related to the temperature of the hot water provided, this thermal flushing with hot water serving to kill bacteria, viruses, etc. Preferably, the thermal flush system is initiated with manual controls due to the possibility of user harm from the scalding water.

Element 01 illustrates various elements of the basin which may be a resin cast sink. Basin 01-1 may be a single compartment sink in accordance with the present invention, and basin 01-2 may be a double compartment sink in accordance with the present invention. Splash guard 01.01 may be an integral splash guard formed from the sink basin 01-1, 01-2. A fluid diverting curb 01.02 may transition into the splash guard 01.01. The front surface, side surface, rear surface and top surface 01.03 may be sloped to drain the respective surfaces. Especially the top surface is sloped to drain to remove contamination and reduce splashing from the faucet and user. Thus, water flow is directed to the drain. The vertical basin back wall is positioned such to facilitate directing the contaminants to the drain.

The slope basin front wall which receives the hand wash and rinse is sloped to reduce splashing from the faucet and the user guiding the water flow to the drain.

The basin drain 01.04 may be positioned away from the user to reduce splash and the present invention may include a concealment lip 01.05. The base drain 01.04 may be closer to the rear wall and the front wall to allow the faucet flow to rinse and remove contamination away from the user, reducing splashing and to avoid splash on the drain

The base assembly 02 may be formed from stainless steel. The basin assembly 02 may include a lower assembly 02.01 having stainless steel cladding and an access door 02.01.A with a continuous hinge. The base assembly 02 may also include a access door thumb screw latch 02.01B to latch the access door 02.01.A and may also include a removable access panel 02.01.C to provide access. The base assembly 02 may also include a base assembly stud 02.01.D.1, a base assembly stud 02.01.D.2 with base retaining bolt, a passage through stud 02.01.E with service protection, bottom shelf 02.01.F, a vibration bumper 02.01.G and a vibration pad 02.01.H.

The base assembly 02 may also include a back splash assembly 02.02 with stainless steel cladding. The back splash assembly 02.02 may include concealed panel clips 02.02.A, a slope top face 02.02.B for dust remediation, and sealant 02.02.C.

The present invention may include an emergency eye wash 03 and a faucet 04. The faucet 04 may include a faucet spout 04.01 including a pressure compensating regulator with a laminar flow water stream with a flow rate per industry regulation, a sensor eye 04.03 to detect the user, a flange 04.04, a back plate 04.05, a locking nut 04.06 and a 90° pipe connection 04.07 for example a US standard NPT ½ inch. The faucet spout 04.01 is a non-aerating laminar flow device. The laminar flow removes turbulence from within the water column and reduces splashing and the non-aerating portion of the nozzle removes air that would otherwise be entrained into the spout nozzle and other biological matter that otherwise would be present.

The faucet spout 04.01 may be positioned closer to the front wall than the back wall to assist the user in the hand wash procedure. The forward location of the faucet assist the person washing and rinsing their hands and arms. If the faucet nozzle is of insufficient length, the user cannot easily rinse their forearms, furthermore the placement of the rinse forward to the user allows for the rinse cycle to capture containments and then move the contamination away from the user and into the drain.

The present invention may include valves 05 which may include a Thermostatic mixing valve 05.01 which may include a maximum set temperature of 48° C., and a temperature mixing valve 05.02.

The Thermostatic mixing valve may include a bypass to allow for pathogens within the faucet to be eliminated with a thermal flush with high temperature sufficient to kill. After the thermal flush, the thermostatic mixing valve is reset to a safe temperature limit to prevent scalding.

The present invention includes a solenoid valve and sensor computer 06 which may include the solenoid valve and sensor computer 06.01, a sensor control wire 06.02, and a transformer power supply 06.03. The faucet under the control of the computer 06 may automatically flush the sink with water at predetermined time intervals to remove any water remaining within the faucet. This facilitates eliminating the possibility of germs growing within the faucet.

The system by the computer 06.01, and manual activation of thermostatic mixing valve 05.01 when used together is a hygienic thermal flush device that will purge the system with scalding water for a length of time sufficient to kill pathogen as determined by the temperature of the hot water and type of pathogen.

The present invention includes a hygienic thermal purge device 07 to provide a manual start with timer and emergency stop; temperature mixing valve, temperature 70° C./160° F. A transformer power supply 07.01 is also illustrated.

The present invention additionally includes a soap dispenser with sensor 08 which may include a dispenser spout 08.01 to dispense the soap, a sensor 08.02 to detect the user, pump and sensor electronics 08.03, a soap reservoir 08.04 a supply hose 08.05 to transport the soap to the dispenser spout 08.01, a sensor control wire 08.06 and a transformer power supply 08.07.

The present invention additionally illustrates an emergency eye/face wash 09 which may include an eye/base wash arm 09.01 with an integral valve, wall mount, and thermo static mixing valve 09.02.

The present invention additionally includes an emergency power circuit 10 to supply electrical duplex power for example, 110V, a check valve 11, a Y strainer 12 which may be stainless steel mesh, and valves 13 which may provide hot and cold water supply.

The present invention may include a water supply 14 which may include a distribution manifold 14.01, a supply hose 14.02 which may include a threaded fitting, a supply hose 14.03 which may include a push fit fitting, and a supply hose 14.04 which may include a threaded and push fit fitting.

The present invention may include a plumbing drain 15 which may include a plumbing drain cover 15.01 and a plumbing P trap 15.02.

The present invention may include a service tag 16 which may include an identification tag 16.01.

The present invention includes a wall assembly 17.

The above embodiments are provided for exemplary and illustrative purposes. Those skilled in the art will appreciate that the present invention is not necessarily limited to such an embodiment. Rather, numerous modifications, and adaptations for particular circumstances, fall within the scope of the invention as herein shown and described.

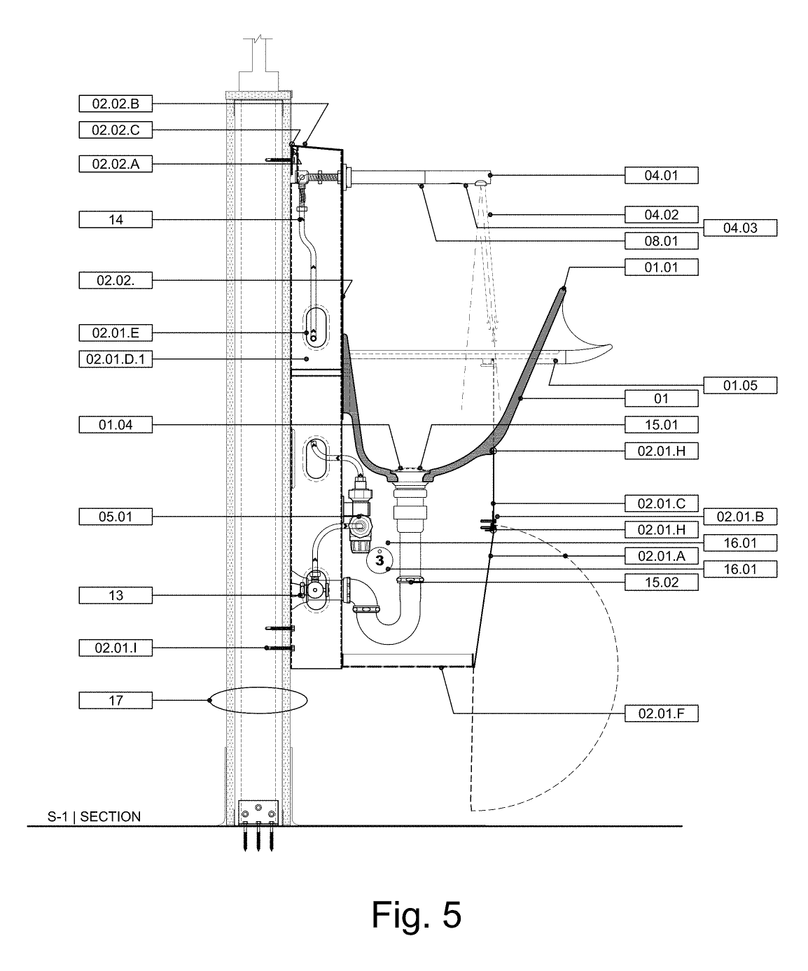

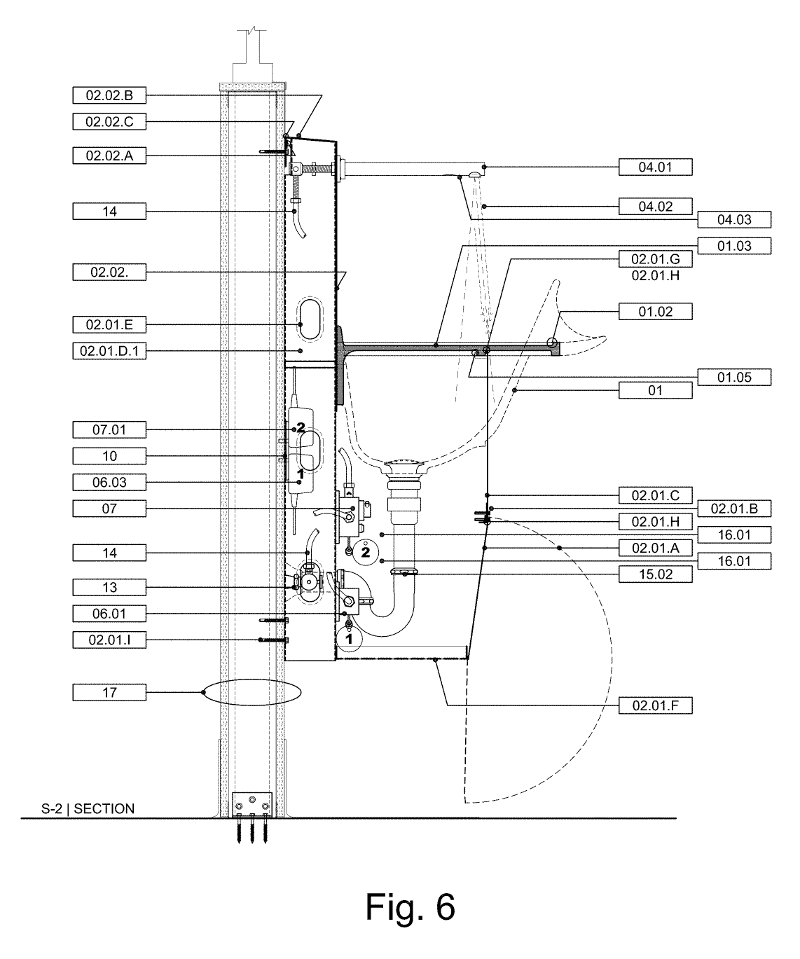

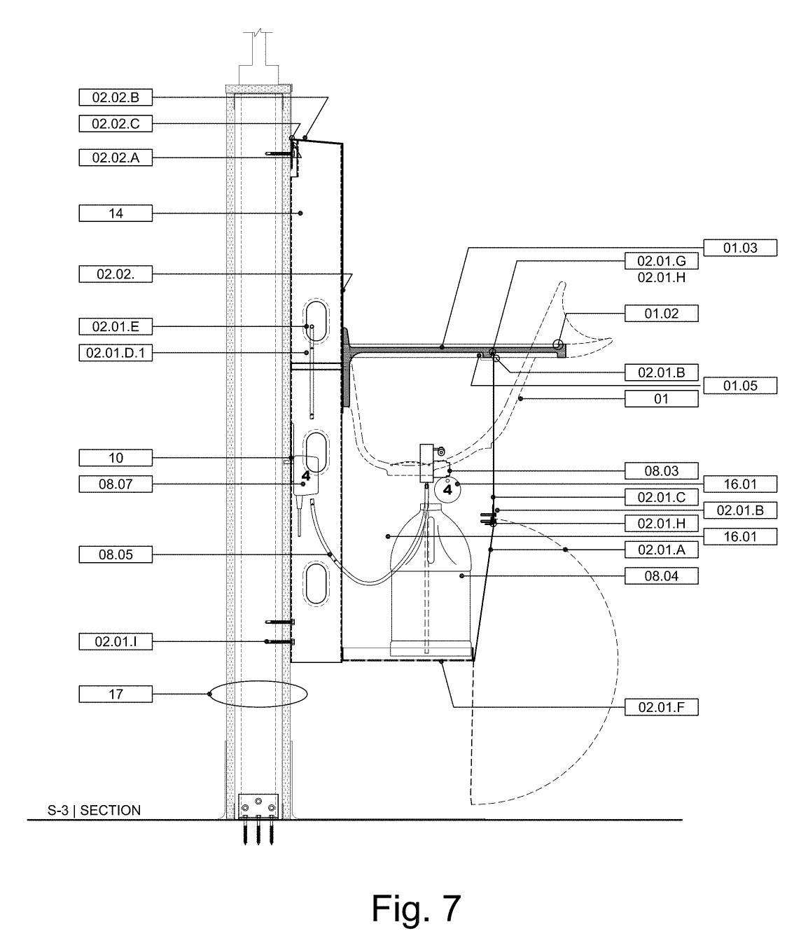

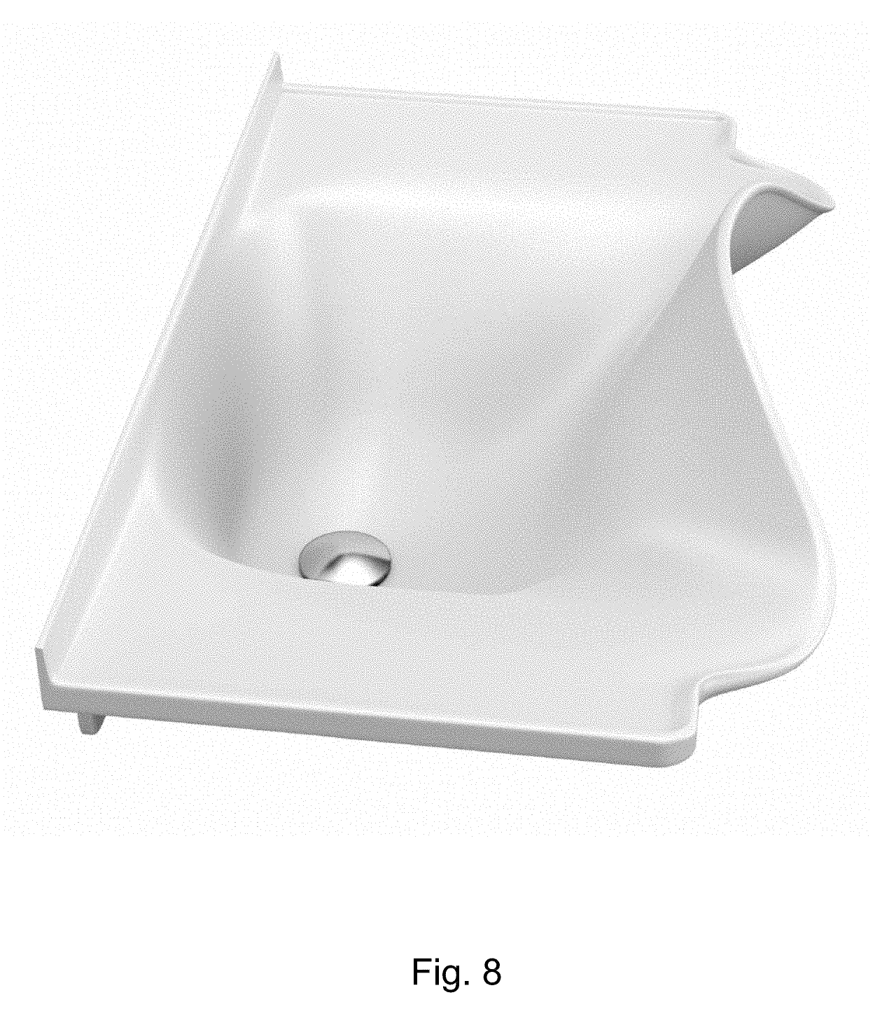

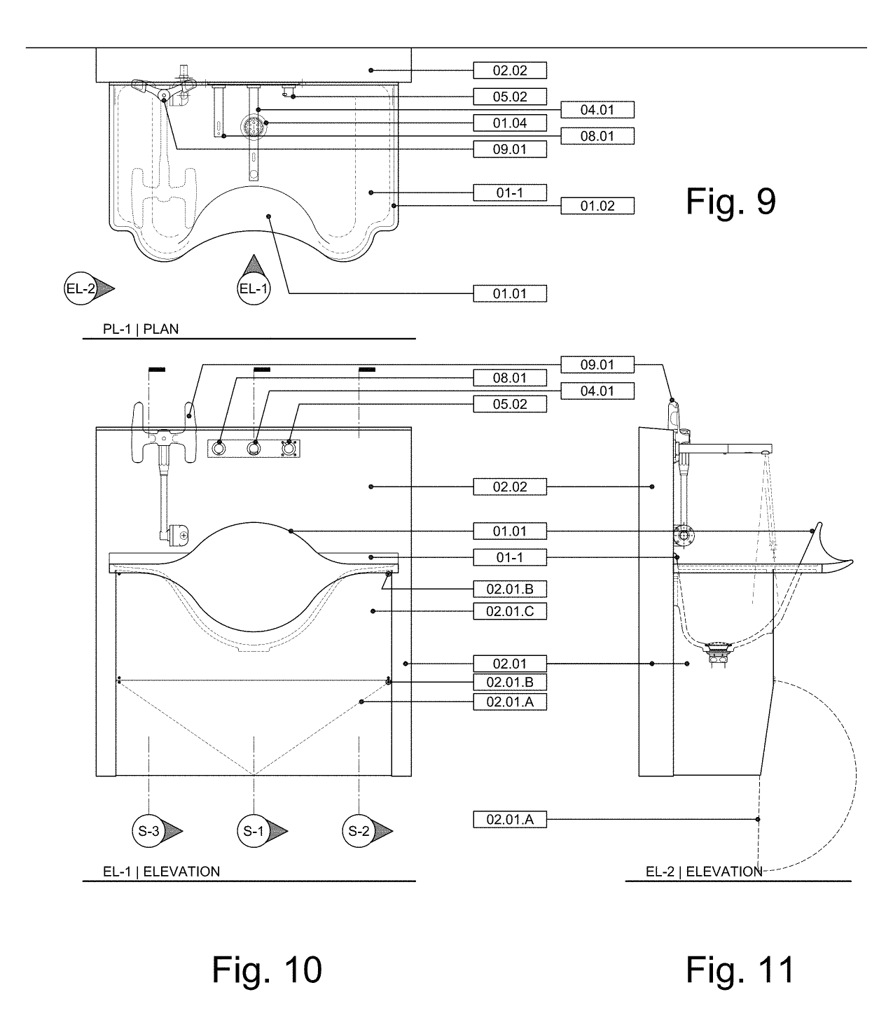

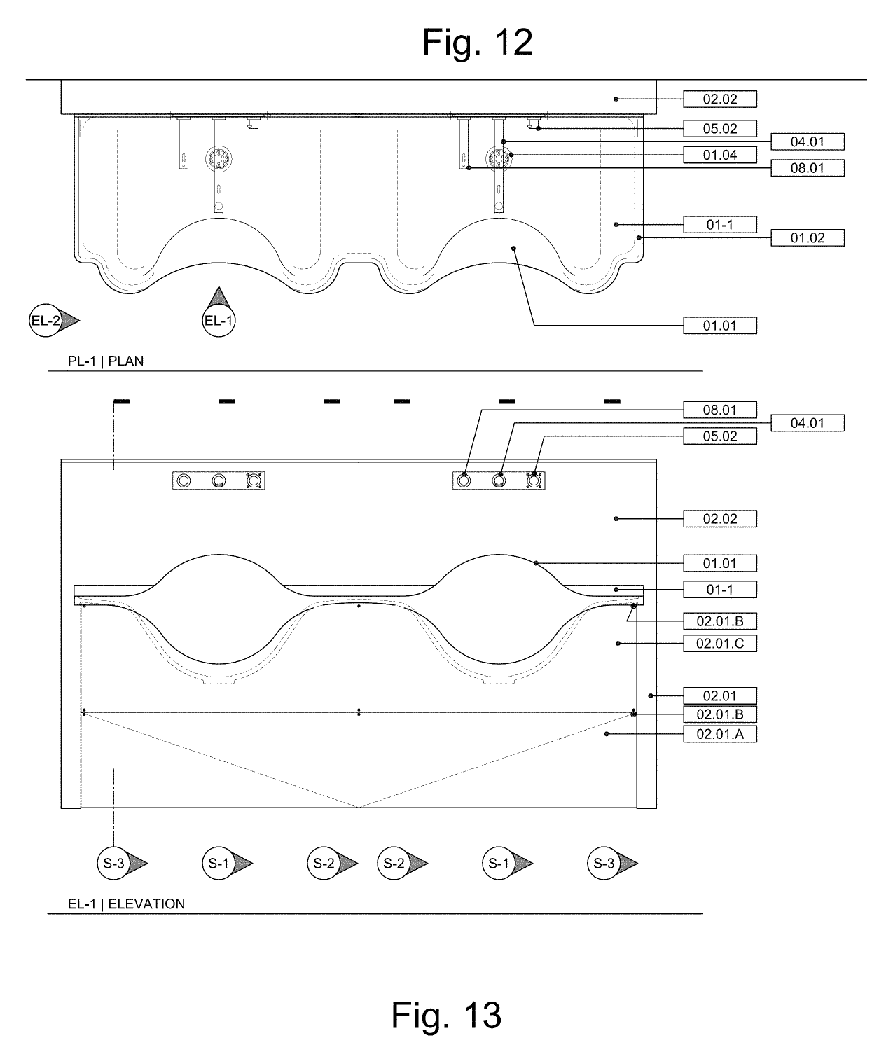

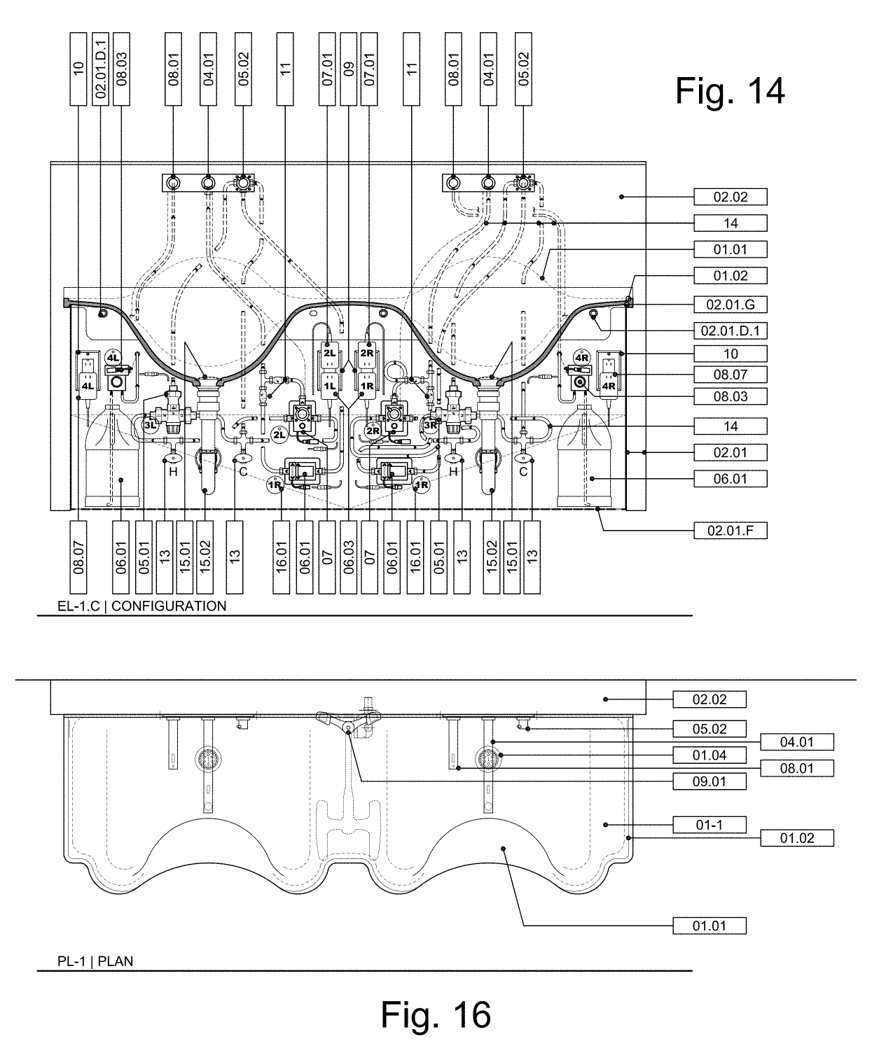

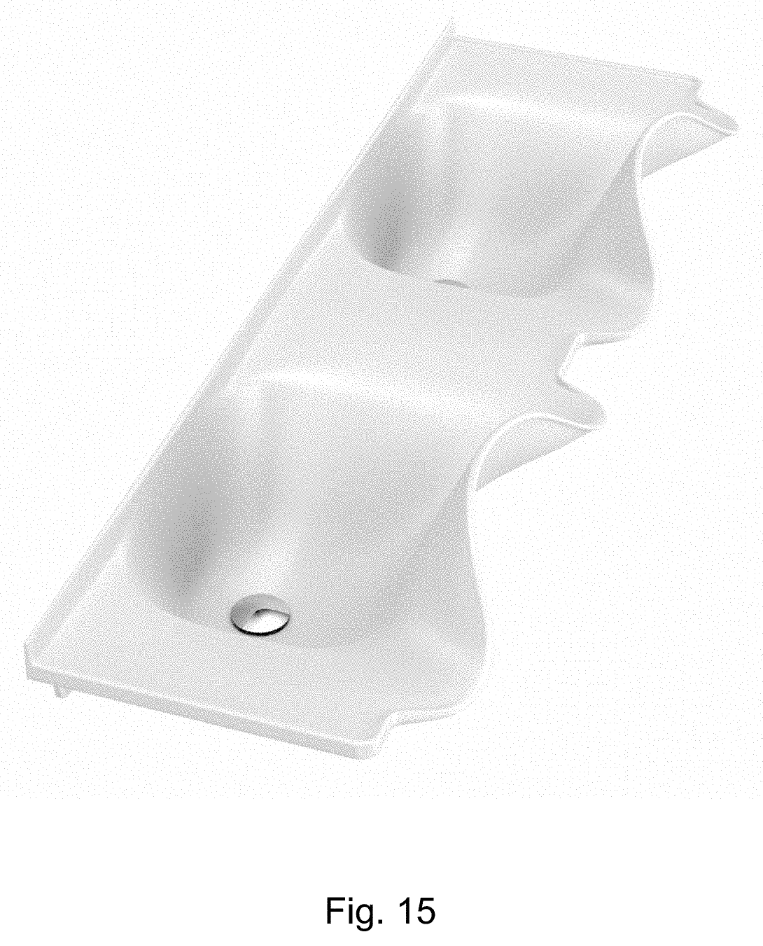

The following tables set forth the components identified by the various reference numbers in the Figures. In connection therewith, it should also be appreciated that the sectional views of the first embodiment in FIGS. 5-7 are also accurately representative of similar sectional views of the second embodiment.

| REV. | ITEM | DESCRIPTION |
|---|---|---|
| | 01 | BASIN: RESIN CAST SINK |
| | 01-1 | BASIN: SINGLE COMPARTMENT SINK |
| | 01-2 | BASIN; DUAL COMPARTMENT SINK |
| | 01.01 | INTEGRAL SPLASH GUARD FORMED FROM |
| | | THE SINK BASIN |
| | 01.02 | FLUID DIVERTING CURB WHICH |
| | | TRANSITIONS INTO THE SPLASH GUARD |
| | 01.03 | SLOPED TO DRAIN FRONT, SIDES, REAR, |
| | | AND TOP SURFACE |
| | 01.04 | BASIN DRAIN LOCATED AWAY FROM THE |
| | | USER TO REDUCE SPLASHING |
| | 01.05 | CONCEALMENT LIP |
| | 02 | BASE: STAINLESS STEEL ASSEMBLY |
| | 02.01 | LOWER ASSEMBLY WITH STAINLESS STEEL |
| | | CLADDING |
| | 02.01.A | ACCESS DOOR WITH CONTINUOUS HINGE |
| | 02.01.B | ACCESS DOOR THUMB SCREW LATCH |
| | 02.01.0 | REMOVABLE ACCESS PANEL |
| | 02.01.D.1 | BASE ASSEMBLY STUD |
| | 02.01.D.2 | BASE ASSEMBLY STUD WITH BASIN |
| | | RETAINING BOLT |
| | 02.01.E | PASSAGE THROUGH STUD WITH SERVICE |
| | | PROTECTION |
| | 02.01.F | BOTTOM SHELF WITH DRAINAGE |
| | 02.01.G | VIBRATION BUMPER |
| | 02.01.H | VIBRATION PAD |
| | 02.02 | BACKSPLASH ASSEMBLY WITH STAINLESS |
| | | STEEL CLADDING |
| | 02.02.A | CONCELED PANEL CLIPS WITH |
| | | RETAINING SCREW |
| | 02.02.B | SLOPED TOP FACE FOR DUST REMEDIATION |
| | 02.02.0 | SEALANT |
| | 03 | EMERGENCY EYEWASH |
| | 04 | FAUCET |
| | 04.01 | FAUCET SPOUT |
| | 04.02 | NEOPERL SLIM PCA CASCADE SLC SSR |
| | | LAMINAR STREAM 2.2 GPM PRESSURE |
| | | COMPENSATING REGULATOR; |
| | | PART NO. 40.8253.010 |
| | 04.03 | SENSOR EYE |
| | 04.04 | FLANGE |
| | 04.05 | BACK PLATE |
| | 04.06 | LOCKING NUT |
| | 04.07 | US STANDARD NPT ½″ 90″ PIPE |
| | | CONNECTION |
| | 05 | VALVES |
| | 05.01 | THERMOSTATIC MIXING VALVE: SET MAX |
| | | TEMPERATURE TO 46° C. |
| | 05.02 | TEMPERATURE MIXING VALVE |
| | 06 | SOLENOID VALVE & SENSOR COMPUTER |
| | 06.01 | SOLENOID VALVE & SENSOR COMPUTER |
| | 06.02 | SENSOR CONTROL WIRE |
| | 06.03 | TRANSFORMER POWER SUPPLY |
| | 07 | HYGIENIC THERMAL PURGE: MANUAL |
| | | START WITH TIMER AND EMERGENCY |
| | | STOP, TEMPERATURE MIXING VALVE, |
| | | MAX TEMPERATURE 70° C./160° F. |
| | 07.01 | TRANSFORMER POWER SUPPLY |
| | 08 | SOAP DISPENSER WITH SENSOR |
| | 08.01 | DISPENSER SPOUT |
| | 08.02 | SENSOR |
| | 08.03 | PUMP & SENSOR ELECTRONICS |
| | 08.04 | SOAP RESERVOIR (BY OTHERS) PROVIDE |
| | | GALLON CAP CONNECTION |
| | 08.05 | SUPPLY HOSE |
| | 08.06 | SENSOR CONTROL WIRE |
| | 08.07 | TRANSFORMER POWER SUPPLY |
| | 09 | EMERGENCY EYE/FACE WASH |
| | 09.01 | SWING-DOWN EYE/FACE WASH ARM WITH |
| | | INTEGRAL VALVE, WALL MOUNT |
| | 09.02 | THERMOSTATIC MXING VALVE |
| | 10 | ELECTRICAL DUPLEX POWER: 110 V ON |
| | | EMERGENCY CIRCUIT |
| | 11 | CHECK VALVE |
| | 12 | Y-STRAINER, STAINLESS STEEL MESH 100 |
| | 13 | VALVES: HOT (H) AND COLD (C) WATER |
| | | SUPPLY |
| | 14 | WATER SUPPLY |
| | 14.01 | DISTRIBUTION MANIFOLD |
| | 14.02 | SUPPLY HOSE: THREADED FITTING |
| | 14.03 | SUPPLY HOSE: PUSH-FIT FITTINGS |
| | 14.04 | SUPPLY HOSE: THREADED AND |
| | | PUSH-FIT FITTING |
| | 15 | PLUMBING DRAIN |
| | 15.01 | PLUMBING DRAIN COVER |
| | 15.02 | PLUMBING P-TRAP |
| | 16 | SERVICE |
| | 17 | WALL ASSEMBLY |

1. A sink to reduce or eliminate infection from being transmitted, comprising;
a front wall;
a back wall being opposed to the front wall;
a pair of opposing side walls connecting the front wall to the back wall;
the front wall, back wall and sidewalls defining a sink cavity;
wherein the cavity includes a drain positioned closer to the back wall than positioned to the front wall.

2) A sink to reduce or eliminate infection from being transmitted as in claim 1, wherein the sink includes a faucet which extends closer to the front wall than the back wall.

3) A sink to reduce or eliminate infection from being transmitted as in claim 1, wherein the sink includes a top wall which is inclined towards a drain of the sink.

4) A sink to reduce or eliminate infection from being transmitted as in claim 3, wherein the back wall is positioned to facilitate contaminants to be directed to the drain.

5) A sink to reduce or eliminate infection from being transmitted as in claim 2, wherein the faucet includes a spout nozzle for non-aerating, laminar flow.

6) A sink to reduce or eliminate infection from being transmitted as in claim 2, where in the faucet includes a automatic flush device to automatically flush the faucet at predetermined intervals.

7) A sink to reduce or eliminate infection from being transmitted as in claim 2, wherein the sink includes a hygienic thermal flush device to purge the spout with scalding water.
